(19) United States
(12) Patent Application Publication
(10) Pub. No.: US 2022/0406461 A1
(43) Pub. Date: Dec. 22, 2022

(54) SYSTEMS AND METHODS FOR ESTIMATING VARIANT-INDUCED DISEASE PENETRANCE AND ESTIMATING PROBABILITY OF DISEASE OCCURRENCE BASED ON THE SAME

(71) Applicant: Vanderbilt University, Nashville, TN (US)

(72) Inventor: Brett M. Kroncke, Nashville, TN (US)

(21) Appl. No.: 17/845,492

(22) Filed: Jun. 21, 2022

Related U.S. Application Data
(60) Provisional application No. 63/212,928, filed on Jun. 21, 2021.

(51) Int. Cl. G16H 50/20; G16H 10/60; G06N 7/08

(52) U.S. Cl. CPC G16H 50/20; G16H 10/60; G06N 7/08

(57) ABSTRACT

Systems and methods for assessing a probability of a disease occurring in a patient based on an estimation of disease penetrance corresponding to a specific genetic variant of interest. An penetrance estimate is determined based on observed penetrance in patient data for the specific variant of interest and observed penetrance for a plurality of other variants that share some commonality with the specific variant of interest. The penetrance estimate is then refined by applying a recursive regression modeling until the penetrance estimate converges towards a final value. The probability of the disease occurring in a patient that has the specific variant of interest is then determined based on the posterior penetrance estimate as determined by the recursive regression modeling.

### RELATED APPLICATIONS

This application claims the priority benefit of U.S. Provisional Patent Application No. 63/212,928, filed Jun. 21, 2021, entitled “A BAYESIAN METHOD TO ESTIMATE VARIANT-INDUCED DISEASE PENETRANCE,” the entire contents of which are hereby incorporated herein by reference.

### STATEMENT OF FEDERALLY SPONSORED RESEARCH OR DEVELOPMENT

This invention was made with government support under grant R00HL135442 awarded by the National Institutes of Health. The government has certain rights in the invention.

### BACKGROUND

The present invention relates to systems and methods for estimating a probability of a disease occurrence in a patient based on genetic data.

### SUMMARY

In some implementations, the systems and methods described herein provide better understanding of the risk of a genetic disease given a specific genetic variation. The approach currently used by the medical genetics community, promulgated by the American College of Medical Genetics and Genomics (ACMG) and the Association for Molecular Pathology (AMP), translates variant data into knowledge of whether that variant can or cannot induce disease; the terminology used is “causative” for disease. In contrast, the framework described in this disclosure integrates variant information (e.g., function, in silico predictions, etc.) into a single annotation ranging from “Benign” to “Pathogenic” (with multiple intermediate classification levels in between). Each additional satisfied criterion raises or lowers the probability the variant is classified “Pathogenic”. This approach is unique because it interprets genetic variants as probabilistically increasing the risk for disease. The systems and methods described herein are centered around a hypothesis that clinically meaningful knowledge is lost in the compression of variant information into a single ACMG annotation. Accordingly, the systems and methods described herein translate variant-specific data into knowledge about the probability of a disease phenotype, the post-test probability of disease or positive predictive value (PPV) for each variant. This is enabled by two major shifts from current thinking: 1) the probability of a genetic disease is estimated by measuring the penetrance of disease (the number of affected carriers divided by the total number of carriers) and 2) variant-specific data is calibrated to this estimate of disease penetrance (or probability/PPV) to better estimate the probability of disease for very rare genetic variants. In some implementations, the systems and methods include translating penetrance in three-dimensional space which is a modification of previous structure-derived features.

In some implementations, variant interpretation is limited to a desire for “yes/no” answers to what is in truth a continuum of probabilities. Other implementations described herein allows for better calibration of variant features because the outcome is not binary and, therefore, contains more information than current strategies. Accordingly, the outcome, or target, enables a more precise interpretation of both variant features and disease probability.

In some implementations, the systems and methods described herein provide the following: (1) Interpretation of all variants (ranging from common to rare) as raising (or not raising) the probability of disease; this probability is measured as the penetrance of disease; (2) Calibrating the estimation of disease probability on penetrance data. This enables an estimate of the probability of disease before observing healthy or sick carriers of genetic variants; and (3) a 3D protein structural feature as a covariate in the method.

The systems and methods described herein are distinct from other technologies that try to understand the consequences of genetic variations in disease-associated genes because those other technologies attempt to classify variants which CAUSE disease whereas the new method determines the probability of disease. Systems and methods described herein are calibrated using penetrance data from many variants, not classification data from many variants. This more precise framework of the relationship between genetic variation and disease probability allows for a more precise understanding of how each individual input feature associates with disease and therefore more informative estimates of disease probability.

In one embodiment, the invention provides a method of assessing a probability of a disease occurring in a patient based on an estimation of disease penetrance corresponding to a specific genetic variant of interest. A set of variants is selected including a specific variant of interest and a database is accessed including genetic and disease data for each of a plurality of individuals. An empirical prior estimate of disease penetrance is calculated based on a number of individuals from the plurality of individuals that have both the disease and at least one variant of the set of variants, and a number of individuals from the plurality of individuals that have at least one variant of the set of variants. A posterior penetrance estimate for the specific variant of interest is then calculated based on the empirical prior estimate and a number of individuals that have the specific variant of interest. A recursive regression modeling is then applied to the posterior penetrance estimate and, in each iteration, an estimated penetrance for the specific variant of interest is fitted to the posterior penetrance estimate and the posterior penetrance estimate is recalculated based on a set of revised variant-specific priors determined based on the regression modeling. The recursive regression modeling continues until one or more defined exit criteria are satisfied (e.g., until the posterior penetrance estimate converges towards a final value). The probability of the disease occurring in a patient that has the specific variant of interest is then determined based on the posterior penetrance estimate as determined by the recursive regression modeling.

Other aspects of the invention will become apparent by consideration of the detailed description and accompanying drawings.

### BRIEF DESCRIPTION OF THE DRAWINGS

FIG. 1 is a flowchart of a method for calculating an estimated variant-specific disease penetrance according to one implementations.

FIG. 2 is a flowchart of a method for determining a disease probability for a patient based on the estimated variant-specific disease penetrance determined by the method of FIG. 1.

FIG. 3 is a block diagram of a computer-based system for identifying a disease probability for a patient using the method of FIG. 2.

FIG. 4 is a histogram of the frequency of variants (y-axis) with different number of individuals diagnosed with Brugada syndrome (x-axis). In this data set, most variants have only a single heterozygote diagnosed with BrS; however, there are over 10 variants with 10 or more heterozygotes diagnosed with BrS.

FIG. 5 is a graph illustrating the frequency of variants (y-axis) with different counts in gnomAD (x-axis). The x-axis is truncated at 350. There are 10 variants with greater than 250 carriers.

FIG. 6 is a graph of the frequency of variants (y-axis) with different observed BrS penetrances (x-axis). IN this data set, most variants have either exactly 0 or exactly 1 observed BrS penetrance, at odds with both the known background rate of BrS in the general public (approximately 1 in 10,000-20,000) and with the extreme rarity of any variant having 100% penetrance.

FIG. 7 is a table of SCNSA variant-specific features used in one example to predict BrS1 penetrance.

FIG. 8 is a series of graphs illustrating penetrance priors informed by variant-specific features. Probability density (y-axis) versus penetrance (x-axis) for three selected SCNSA variants where peak current, penetrance density, and in silico classification are known. Numbers are affected and unaffected individuals reported are presented for each variant. Penetrance priors are low for c.3922C>T, moderate for c.4978A>G, and higher for c.2632C>T. When variant-specific data are known, the penetrance estimate is adjusted to reflect the penetrance probability consistent with variants with similar features.

FIG. 9 is a graph of a Bland-Altman plot between EM prior and EM posterior mean penetrances for all SCNSA variants. To assess performance of the EM prior, the Bland-Altman plot compares the mean BrS1 penetrance estimated from the EM prior and from the EM posterior, the y-axis is the difference between the two and the x-axis is the average between the two. For each plotted point, both color and radius indicates the log₁₀ of the total number of heterozygotes presented in the dataset. The relatively consistent scatter about y=0 suggests no systematic biases present in the EM prior mean BrS1 estimates.

FIG. 10 is a table of weighted R² values from EM prior means to Empirical/EM posterior means.

FIG. 11 is a graph illustrating prior mean BrS1 penetrance reflecting the protein topology of Naᵥ1.5 and the predicted mean BrS1 penetrance from the converged expectation maximization algorithm of FIG. 1. The line across the plot is a predicted mean BrS1 penetrance averaged over 30 neighboring variants. Topology diagram is shown above with transmembrane helices indicated by yellow lines and membranes indicated as a grey rectangle. Note the four largest, distinct peaks correspond to the four structure, transmembrane domains of the channel with an especially steep peak at the selectively filter and pore. Though estimated distances in three-dimensional space between residues is used to construct the BrS1 penetrance density, structural data are not explicitly used in the BrS1 penetrance prior and so the recapitulation of the structure is not assured.

FIG. 12 is a graph of sample BrS1 penetrance prior 95% credible intervals for each of a plurality of different variants. The y-axis lists SCN5A variatns with more than one heterozygote in the dataset plotted with prior 95% credicable intervals and mean posteriors (black rectangles). A model to the SCN5A protein product, Naᵥ1.5, is shown adjacent to the graph with regions highlighted in colors corresponding to the variant prior 95% credible intervals shown in the graph, which are analogos to the penetrance probability distributions shown on the y-axes in FIG. 8. Variants near the D-III pore selectively filter have a much higher prior and posterior BrS1 penetrance compared to residues near the D-III/D-IV linker. This is expected since the selectively filter pore helices contain the most compacted region of the protein and also is responsible for the ion conduction and is therefore most sensitive to substitution. In fact, the highest density of variants with non-zero BrS1 penetrance lie at this depth in the membrane.

FIG. 13 is a graph illustrating that SCNSA pathogenic and benign variants cluster in space. Rate of variants with high BrS1 penetrance (>20%, blue) or low BrS1 penetrance (<10%, red) in a model of the SCNSA protein product. Each bar represents a histogram of variants associated with each disease within a 5 Å slice within the membrane (divided by the total number of residues within the slice), boxes at each of the four corners represent residues not modeled (only 33 residues were not modeled in the extracellular loops). There is a relative paucity of low BrS1 penetrance variants within the structured transmembrane region and the relative abundance of high BrS1 penetrance in the same region. The rate of high BrS1 penetrance variants is higher in the extracellular half of the protein molecule likely due to more compacting of residues in the top half of the pore domain as well as proximity to the ion selective element (selectivity filter). Amino acid substitutions in these regions therefore more often have a disruptive influence.

FIG. 14 is a graph of BrS1 penetrance probability versus penetrance for the empirical prior.

FIG. 15 is a graph of estimated coverage rates for each SCNSA variant versus sampled true penetrance. Coverage rate was calculated as defined above. Color and radius indicate the log₁₀ of the total number of heterozygotes present in the dataset. The tuning parameter Equation 4 was set to v=7. There is overcoverage (greater than 95%) for variants with high and low BrS1 penetrance indicating an overestimate of the variance.

FIG. 16 is a graph of estimated coverage rates for each SCNSA variant versus sampled true penetrance. Coverage rate was calculated as defined above. Color and radius indicate the log₁₀ of the total number of heterozygotes present in the dataset. The tuning parameter Equation 4 was set to v=14. There is overcoverage for the majority of variants, though some variants are now outside the 95% credible interval.

FIG. 17 is a graph of estimated coverage rates for each SCNSA variant versus sampled true penetrance. Coverage rate was calculated as defined above. Color and radius indicate the log10 of the total number of heterozygotes present in the dataset. The tuning parameter Equation 4 was set to v=19. Overcoverage is reduced especially for residues with very low or very high BrS1 penetrance, indicating an appropriate estimate of variance.

FIG. 18 is a graph of estimated coverage rates for each SCNSA variant versus sampled true penetrance. Coverage rate was calculated as defined above. Color and radius indicate the log10 of the total number of heterozygotes present in the dataset. The tuning parameter Equation 4 was set to v=99. Variant undercoverage is much more prevalent and distributed evenly across variants with low to high BrS1 penetrance indicating an overestimate of variance.

FIG. 19 is a histogram of BrS1 penetrance imputed EM prior means and associated upper and lower bounds to 95% credible interval from pattern mixture models. Plotted are BrS1 mean penetrances from imputed EM priors (“Predicted”, green) and upper (red) and lower (blue) bounds to associated 95% credible intervals from those imputed EM priors.

FIG. 20 is a graph of a Bland-Altman plot between EM posterior mean BrS penetrances and observed BrS penetrances for the SSCN5A variants with at least 15 heterozygotes. The relatively narrow spread along the y-axis suggests reasonable agreement between the two estimates of BrS penetrance. With the cutoff of at least 15 heterozygotes, there are relatively few variants with an expected penetrance of greater than 10%.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

A major challenge emerging in genomic medicine is how to assess best disease risk from rare or novel variants found in disease-related genes. The expanding volume of data generated by very large phenotyping efforts coupled to DNA sequence data presents an opportunity to reinterpret genetic liability of disease risk. Here we propose a framework to estimate the probability of disease given the presence of a genetic variant conditioned on features of that variant. We refer to this as the penetrance; the fraction of all variant heterozygotes that will present with disease.

In various specific examples described herein, we demonstrate this methodology using a well-established disease-gene pair, the cardiac sodium channel gene SCNSA and the heart arrhythmia Brugada syndrome. From a review of 756 publications, we developed a pattern mixture algorithm, based on a Bayesian Beta-Binomial model, to generate SCNSA penetrance probabilities for the Brugada syndrome conditioned on variant-specific attributes. These probabilities are determined from variant-specific features (e.g. function, structural context, and sequence conservation) and from observations of affected and unaffected heterozygotes. Variant functional perturbation and structural context prove most predictive of Brugada syndrome penetrance.

The clinical implications for genetic variants, even definitively pathogenic variants, can vary strikingly across individuals. Lack of evidence to estimate the probability of disease from identified genetic variants, especially rare variants, presents a major barrier to integrating genotype information into clinical care. Here we advance an approach to estimate the penetrance, or positive predictive value of the discovery of a genetic variant, in service of advancing the use of genetic information in personalized medicine.

A major barrier to integrating genotype information into clinical care is accurately linking genetic variants to disease risk. As cheap whole genome, exome, and gene panel sequencing becomes more widely used, the genetics community frequently observes novel, ultra-rare variants—ones carried by a single or few (often related) individuals. Indeed, most variants found in large population genome sequencing efforts are novel or ultra rare. The number of possible single nucleotide variants in the human genome is in the billions; the number of variants becomes uncountable if insertion and/or deletions (indels) are included. The majority of these discovered variants will never be observed in a sufficient number of heterozygotes to ascertain a causal link with disease. In addition to finding rare variants, large-scale genetic sequencing efforts taking place around the world are identifying greater numbers of individuals, ostensibly unaffected, who carry variants previously thought to be disease-inducing. As a consequence of insufficient heterozygote counts and conflicting annotations, many diagnostic laboratories annotate such variants as “Variants of Uncertain Significance” (VUS), despite more confident past assessments of “Likely Pathogenic” or “Pathogenic.”.

To help assess the impact of genetic variants, the American College of Medical Genetics and Genomics (ACMG) suggests integrating multiple sources of information including population, functional, computational, and segregation data to classify variants. This is consistent with a continuous, Bayesian framework where each additional satisfied classification criterion modifies the probability a variant is causative for disease (pathogenic) or not (benign). Given the resulting probabilities, a final classification can be made into one of the five categories commonly used to distinguish variants including, for example, “benign”, “likely benign”, “variant of uncertain significance”, “likely pathogenic”, or “pathogenic”. However, a remaining challenge even after classification is that the clinical implications for definitively pathogenic variants can vary strikingly across individuals, including variable expressivity and incomplete penetrance. We attempt here to address one aspect of this clinical variability by developing a method to estimate variant-induced disease risk.

In this study, we sought to develop a method to estimate the probability of disease given variant-specific information—which we refer to as the penetrance of a variant—and we also provide the uncertainty for that estimate. The pathogenicity of a variant for a specific individual at a given point in time is binary but unknown. This pathogenicity may have a time dependence such as for diseases which present later in life. Penetrance is one metric that captures the degree to which the pathogenicity will manifest as a human phenotype such as a disease or a trait. We provide posterior probability estimates of the penetrance, asymptotic with respect to age, which can be thought of as the positive predictive value of disease given the known variant information. We also provide a 95% credible interval that represents the uncertainty in that estimate. Our method relies on “borrowing strength” or sharing information across variants to produce variant-specific, quantitative penetrance estimates even in the absence of a large number of heterozygotes. These estimates can be especially informative for interpreting rare and novel variants.

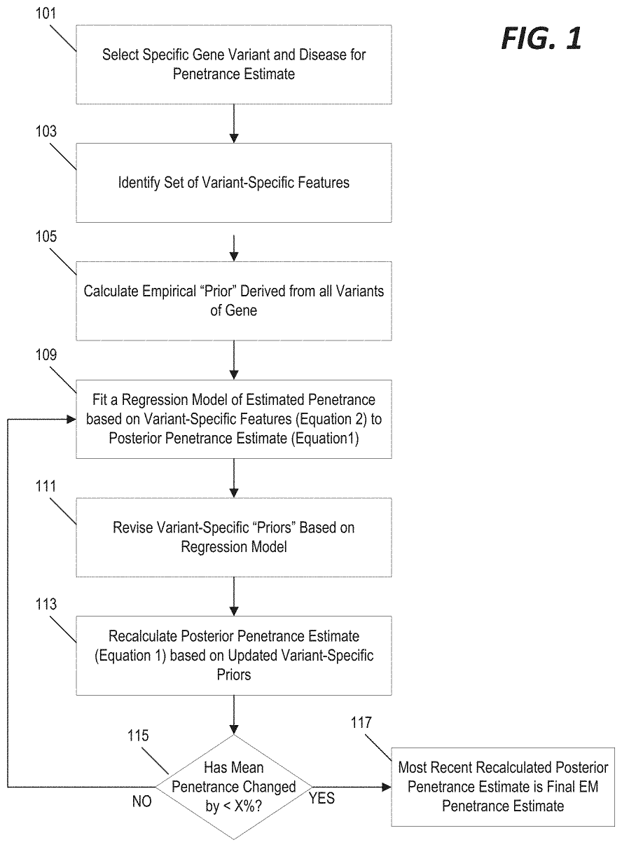

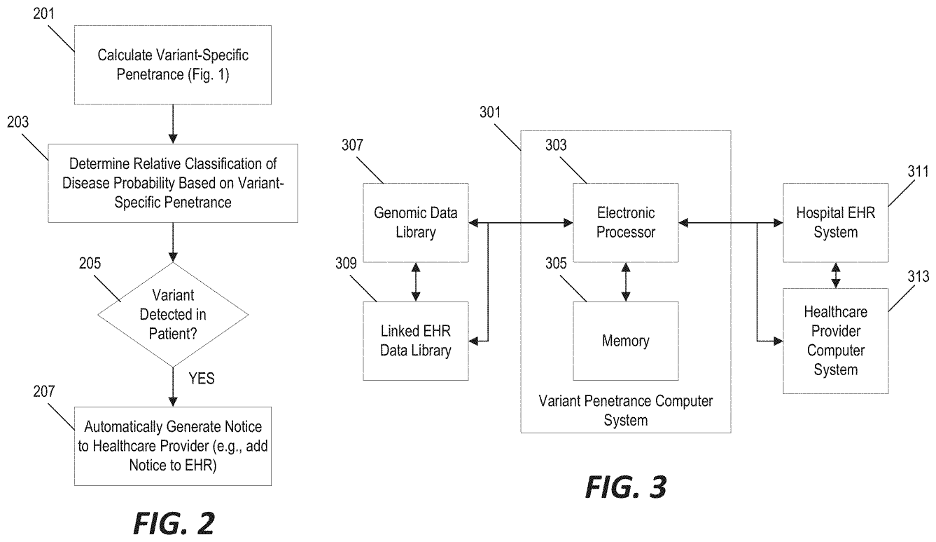

FIG. 1 illustrates an example of a method for estimating a disease penetrance for a specific variant of a gene. Penetrance is defined as the fraction of individuals who carry a variant that also present with a disease. This can be extracted from literature reports, electronic health records, or other databases when multiple variant heterozygotes have been reported. Although we cannot observe actual penetrance for any given variant, we can estimate penetrance for each variant of a gene as the average posterior penetrance denoted as the following:

$$Mean Posterior {Penetrance}_{i} = \frac{{α}_{i ,} + {α}_{i , prior}}{{α}_{i ,} + {α}_{i , prior} + {β}_{i} + {β}_{i , prior}} \qquad ( 1 )$$

where α is the number of variant heterozygotes diagnoses with a particular disease and β is the number of unaffected heterozygotes of the same variant, i is the specific variant in question and mean posterior penetrance is the estimated penetrance or probability of disease. As the total number of observed heterozygotes increases, the estimated penetrance converges to the traditional definition. The mean posterior penetrance can be thought of as a shrunken estimate of the observed penetrance, especially for variants with small numbers of known heterozygotes.

To generate “priors” from available data, the method of FIG. 1 uses an algorithm based on the “expectation maximization” (EM) concept. This EM algorithm is an iterative technique comprising three steps: (1) calculate the expected penetrance from an empirical Bayes penetrance model, (2) fit a regression model of our estimated penetrance on variant-specific characteristics by maximum likelihood (e.g., using Equation (2) below), and (3) revise the estimate of the penetrance prior using fit from the regression analysis until convergence criteria are satisfied. Equation (2) presents an example of a method for calculating a penetrance estimate for variant-specific factors including peak current, penetrance density, and other in silico variant classifiers.

Penetrance Estimateᵢ=β₀+β₁(Peak Current)ᵢ+β₂(Penetrance Density)ᵢ+Σₙβ_{i,n}(In Silica Variant Classifiers)_{i,n}+εᵢ (2)

The fitted model is then used to generate an updated prior distribution and, by addition of observed cases (i.e., heterozygotes with disease) and controls (i.e., heterozygotes without the disease) for each variant, a subsequent posterior expected penetrance. The new EM priors are unique to each variant and replace αₚᵣᵢₒᵣ and βₚᵣᵢₒᵣ in equation (1) for the next iteration. The updated posterior penetrance is then used to build a new fitted model and further refine the posterior expected penetrance. The procedure is iterated until it converges to a maximum likelihood solution. Although the example of FIG. 1 discusses the use of a linear regression model to fit estimated penetrance to posterior penetrance estimate, other approaches may be uses in other implementations including, for example, other types of regression modeling and/or machine learning mechanisms (e.g., “interacting terms”).

As illustrated in FIG. 1, the method begins by selecting or identifying a specific gene variant and a specific disease for further study (i.e., a specific variant-disease pairing for which penetrance will be estimated) (step 101). Based on the selected variant, a set of variant-specific features are then identified (i.e., features that medical records indicate correspond to patients exhibiting the specific variant or to the variant itself) (step 103). Next an Empirical “Prior” probability is derived from information available for all variants of the gene in question (e.g., Empirical prior is calculated as the total number of individuals that exhibit any variant on the gene in question and also present the disease divided by the total number of individuals that exhibit any variant on the gene in question (regardless of whether they have the disease)) (step 105). The empirical prior is a single αₚᵣᵢₒᵣ value and a single βₚᵣᵢₒᵣ value for all i variants. In some implementations, the variants used to calculate the empirical prior art selected only from a subtype of variant, a missense, or in-frame insertions/deletions. In some implementations, the model illustrated in FIG. 1 is used for variants where the covariates used are not all the same—in other words, variants are treated separately based on relatability of variant features. For example, in some implementations, missense variants and non-sense variants (e.g., no protein is produced) are not used in the same analysis.

Next a regression model is used to fit an estimated penetrance based on variant-specific features (e.g., the result of Equation (2)) to the posterior penetrance estimate of Equation (1) (step 109). The variant-specific priors (i.e., the probability correlation of the variant-specific features to the disease) are then revised based on the regression model (step 111) and the posterior penetrance estimate (equation (1)) is recalculated based on the updated variant-specific priors (step 113). In other words, after the first iteration, αₚᵣᵢₒᵣ and βₚᵣᵢₒᵣ in the empirical model are replaced with new variant-specific values of α_{i,prior} and β_{i,prior} based on the regression model. After each subsequent iteration, α_{i,prior} and β_{i,prior} are replaced with updated values based on the regression model. This process is repeated iteratively by fitting a regression model of the estimated penetrance based on variant-specific features (e.g., equation (2)) to the updated posterior penetrance estimate until one or more convergence criteria are met (e.g., until an iteration fails to change the calculated mean penetrance by more than a defined percentage) (step 115). When the convergence criteria (or criterion) are satisfied, then the most recent version of the recalculated posterior penetrance estimate is the final EM penetrance estimate for the specific variant (step 117).

In some implementations, the estimated disease penetrance for the specific variant can be used to identify and/or quantify risk conditions for the disease in patients. For example, in the method of FIG. 2, a variant-specific disease penetrance is calculated (e.g., using the method of FIG. 1) (step 201) and, based on the magnitude of the variant-specific penetrance, a relative classification of disease probability is assigned to the specific variant (step 203). In some implementations, the relative classification is placed in one of a defined number of classification categories (e.g., benign, likely, benign, variant of uncertain significance, likely pathogenic, or pathogenic). In other implementations, a numeric score is assigned to the variant based on the estimated disease penetrance as an indication of relative probability of the disease occurring in patients that have the specific variant. After a relative classification of disease probability is assigned to the specific variant, a system can be configured to monitor for occurrences of the specific variant in patients (e.g., based on genomic data added to the patient's electronic health record). In some implementations, a computer-based system can be configured to detect an occurrence of the specific variant in a patient (step 205) and to then automatically generate a notice to a healthcare provider (step 207). In some implementations, the automatically generated notice is informative in nature and is intended to inform the healthcare professional of a potential risk for a disease. In other implementations, the system may be configured to take other actions in response to detecting an occurrence of the variant including, for example, automatically scheduling/initiating a treatment protocol associated with the disease in response to detecting the variant.

FIG. 3 illustrates an example of a computer-based system configured to determine an estimated disease penetrance for a specific variant (e.g., using the method of FIG. 1) and for automatically performing some mitigative action in response to detecting occurrences of the variant based on an estimated risk classification assigned to the variant (e.g., using the method of FIG. 2). A variant penetrance computer system 301 includes an electronic processor 303 and a non-transitory, computer-readable memory 305. The memory 305 stores data and instructions that are accessible and executable by the electronic processor 303. Execution of the instructions by the electronic processor 303 provides functionality of the variant penetrance computer system 301 including, for example, the functionality described herein. The variant penetrance computer system 301 is communicatively coupled to a genomic data library 307 that includes a repository of anonymized genetic data. The genomic data library 307 is linked to an EHR data library 309 that also includes anonymized data for a plurality of patients (for research purposes). In order to facilitate research, the anonymized EHR data in the EHR data library 309 is linked to the genomic data in the genomic data library 307 so that phenotype data can be matched with corresponding genotype data.

The variant penetrance computer system 301 analyzes and processes data from the genomic data library 307 and the linked EHR data library 309 in order to calculate an estimated disease penetrance for the specific variant (e.g., using the method of FIG. 1). In some implementations, the system is configured to update the calculation of disease penetrance for the specific variant as more heterozygotes with and without the disease are added to the database (e.g., by periodically performing an updated calculation or by automatically performing the updated calculation in response to determining that a threshold number of new heterozygotes have been added to the database). The variant penetrance computer system 301 is also communicatively coupled to a hospitals EHR system 311 (or other health record system), detects occurrences of the specific variant and then interacts with a healthcare provider's computer system 313 in response to detecting the variant based at least in part on the risk for the disease estimated for the specific variant.

To further illustrate this approach, below are a series of examples applying the method of FIG. 1 using a rare cardiac arrhythmia disorder Brugada Syndrome (BrS1 [MIM:) 601144]), which is linked to rare loss-of-function variants in the cardiac sodium channel SCN5A. These variants most commonly act by altering peak sodium current, a parameter of sodium channel function that is readily assessed using in vitro methods. By quantitatively integrating multiple features, including in vitro functional experiments, information about the three-dimensional protein structure, and previously published variant-classifiers, such as PolyPhen-2 and PROVEAN, we estimate the BrS1 penetrance attributable to individual SCN5A variants. The resulting priors, imputed from these predictive features, can be readily interpreted as hypothetical observations of unaffected and affected heterozygotes.

Variants in SCN5A have been associated with BrS1 since 1998, some variants affecting almost all known heterozygous individuals, some variants conferring only modestly increased risk, and others have no influence on arrhythmia presentation. SCN5A variants that do not influence the gene in any way do not predispose or protect against BrS1, e.g. many synonymous variants. These variants therefore have a relatively low penetrance of the arrhythmia, similar to the general population. SCN5A variants that produce no sodium current result in a higher fraction of heterozygotes presenting with BrS1, much higher than in the general population. However, BrS1 presentation, as for nearly all inherited diseases, is not homogeneous even amongst heterozygotes of SCN5A haploinsufficiency alleles. In fact, even highly penetrant variants such as R878C and E1784K still leave some heterozygotes unaffected: 100% penetrance is extremely rare.

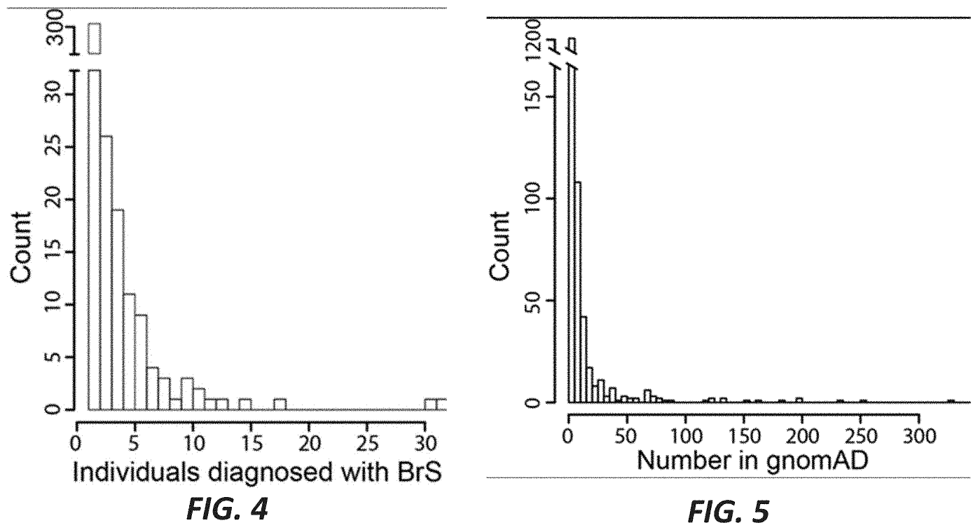

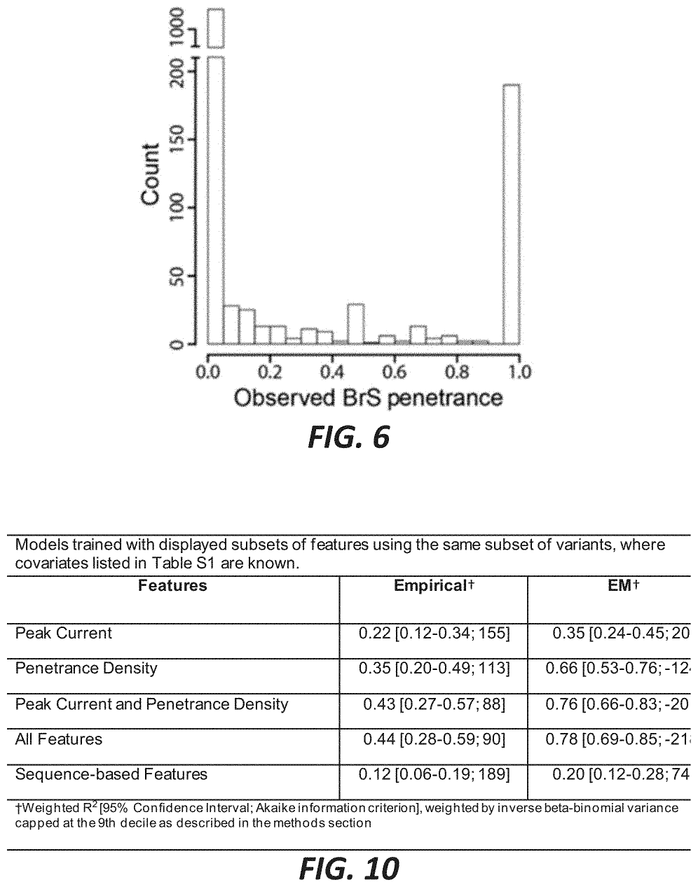

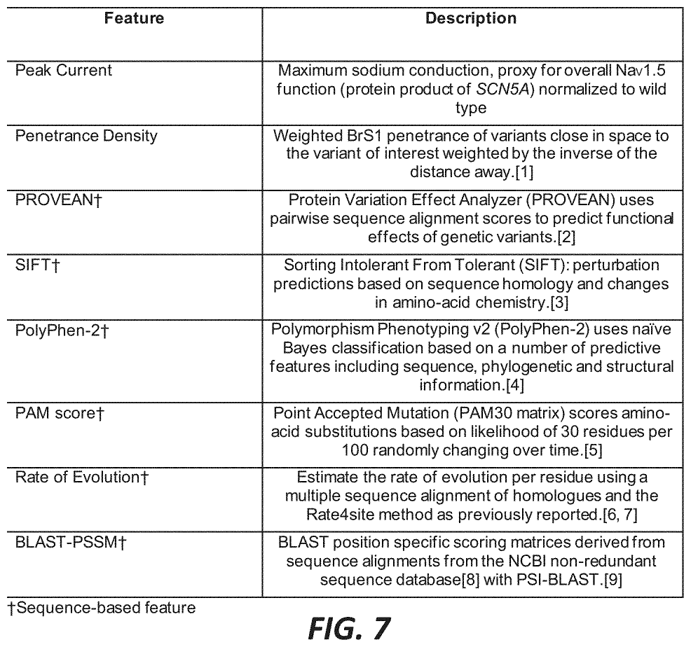

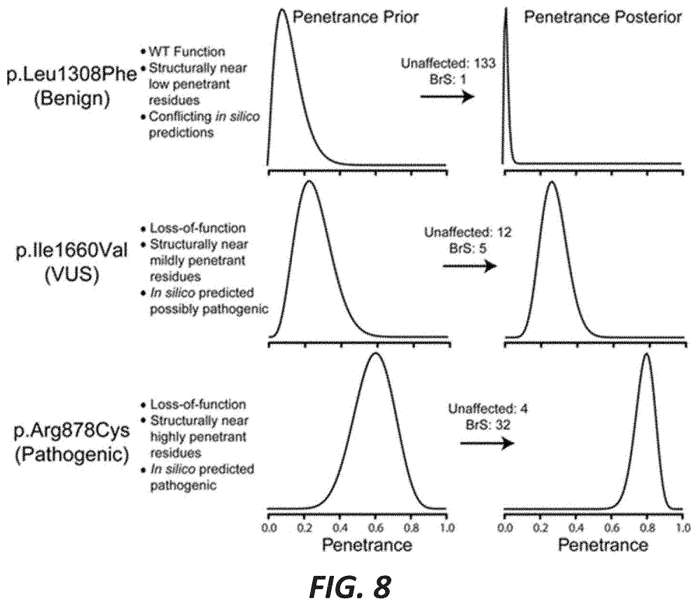

Our hypothesis is that variant-specific features (e.g. variant-induced changes in function and location in structure) contain information equivalent to clinically phenotyping heterozygotes and can therefore be used to inform the prior distribution in a Bayesian framework. This prior distribution is combined directly with clinically phenotyped heterozygotes (the likelihood function) to produce more accurate estimates of disease risk probability (posterior penetrance; FIG. 8) via Bayes theorem. To demonstrate this approach, we developed an expectation maximization approach (EM) and applied it to a previously generated dataset of SCN5A features and BrS1 phenotype counts (supplemented with reports published within the last year) to estimate BrS1 penetrance using SCN5A variant-specific features. This process yielded a total of 1,439 unique variants with at least 1 observed heterozygote, BrS1 was diagnosable in 857 individuals heterozygous for 387 unique variants (see, FIGS. 4-6). BrS1 penetrance priors informed by the predictive features listed in the table of FIG. 7 adjust and narrow the uncertainty, as shown in FIG. 8.

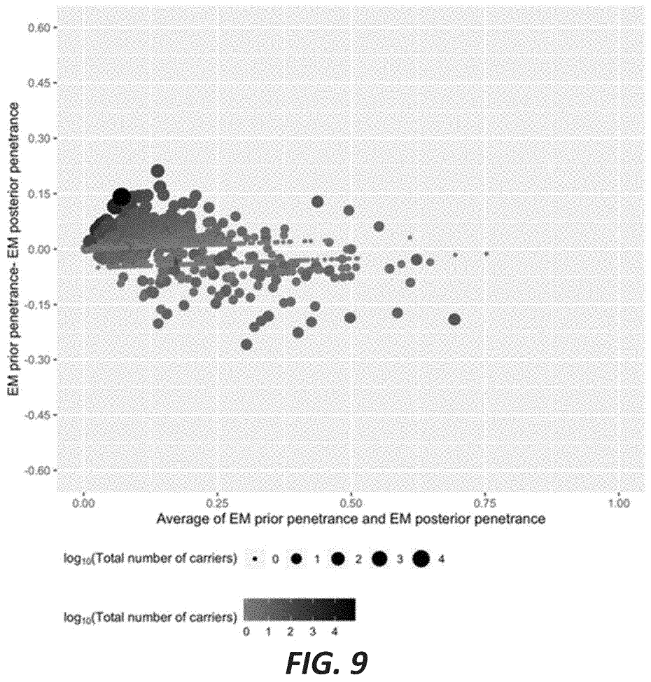

Precision and accuracy of BrS1 penetrance priors. To evaluate performance over the distribution of BrS1 prior penetrances (FIG. 19), we plotted the difference between prior mean and posterior mean BrS1 penetrance as a function of the average between the two estimates (FIG. 9). The resulting Bland-Altman difference plot seen in FIG. 9 indicates scatter evenly distributed with under and over predicted BrS1 penetrance as a function of prior mean penetrance. This suggests the predictive priors are reasonably calibrated and have no systematic biases in the range of BrS1 mean penetrance estimated. We additionally compared linear regression models trained on a limited subset of features/covariates with the BrS1 mean posterior,

$$\frac{BrS 1 cases + {α}_{prior}}{total heterozygotes + {α}_{prior} + {β}_{prior}}$$

(where αₚᵣᵢₒᵣ and βₚᵣᵢₒᵣ are the tuning parameters for the beta-binomial distribution and are set equivalent to the number of affected and unaffected individual heterozygotes in the prior), as the dependent variable; both empirical and EM priors were evaluated as indicated in the table of FIG. 10. Peak current and penetrance density contain orthogonal information as can been seen by the differences in coefficient of determination, R², for models built using each or both predictors (see, table of FIG. 10). The relatively small improvement in R² when all predictors are included suggests most information contained in the sequence-based predictive features is recapitulated by both peak current and penetrance density.

Inclusion of individuals from gnomAD. Individuals in gnomAD are mostly unaffected, given the rarity of BrS; however, the data available from that resource could be contaminated with individuals presenting with BrS, though likely at or near the rate in the general public. To test the sensitivity of our results to this type of misclassification, we randomly switched individuals from unaffected (gnomAD) to BrS cases for each variant and examined the change in penetrance due to misclassification. We did this with 24 and 240 misclassified cases. With 24 misclassifications, the median rate of penetrance change is 0.4% and the expected number of variants with a penetrance change is 6. The average mean absolute difference in penetrance change is 0.02% (first quartile of 0.0014% and third quartile of 0.02%). With 240 misclassifications, the median rate of penetrance change is 2%, and the expected number of variants with a penetrance change is 28. The average mean absolute difference in penetrance change is 0.2% (first quartile of 0.1% and third quartile of 0.3%). These results suggest minimal influence of small or modest misclassification rates on penetrance estimates.

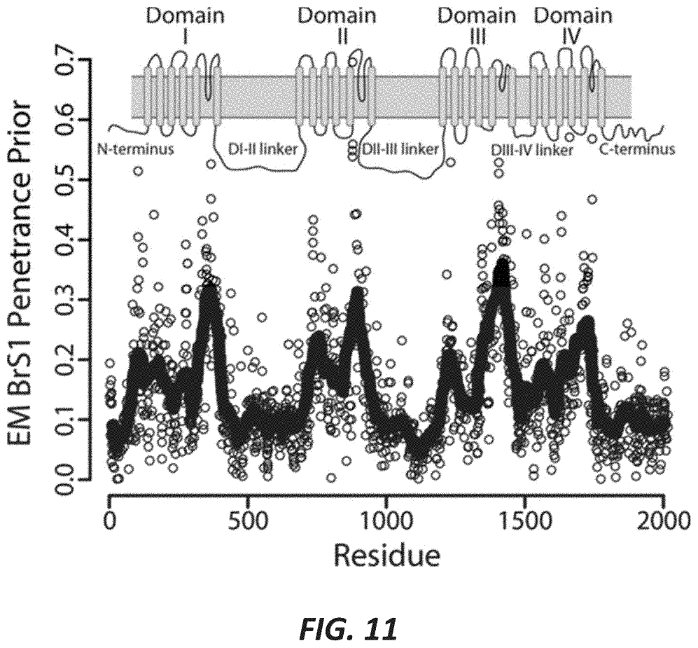

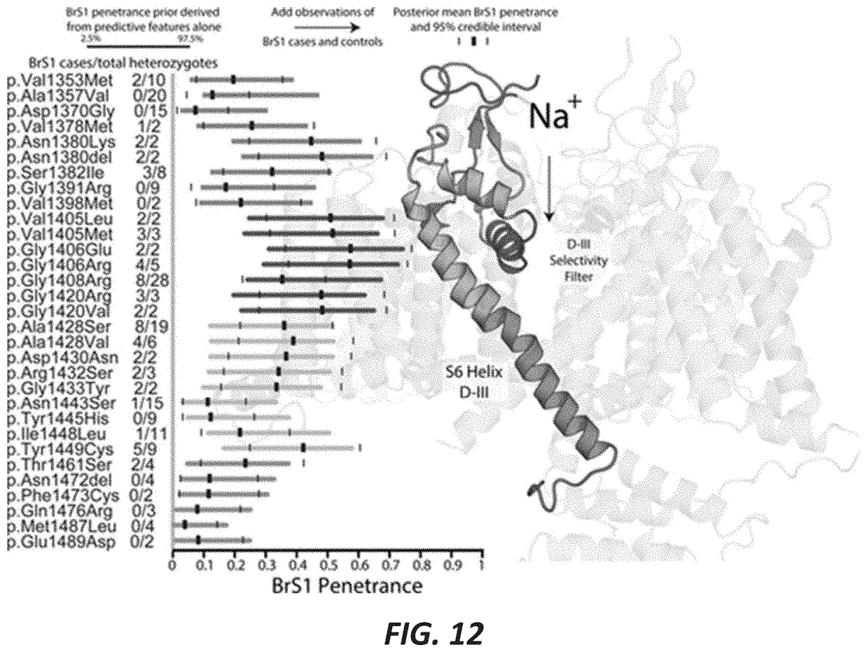

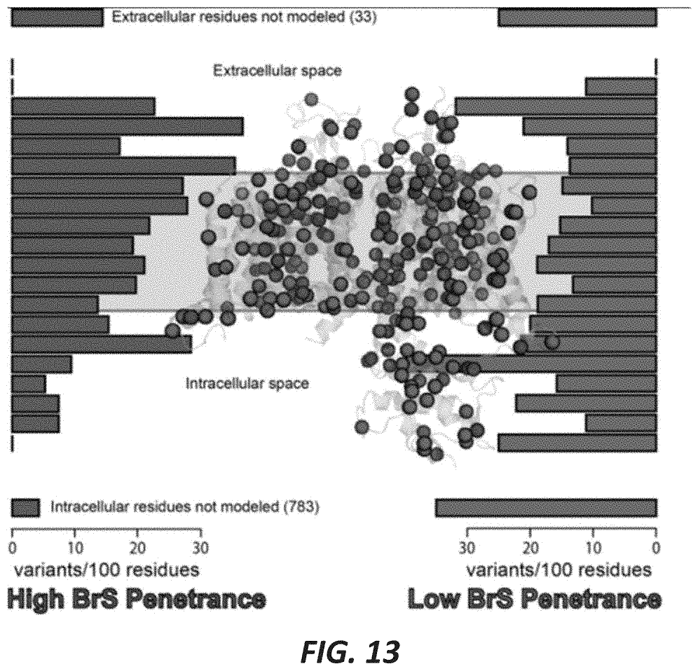

Structure and peak current improve prediction of penetrance. The resulting prior BrS1 mean penetrance estimates reflect the known topology of Naᵥ1.5 (protein product of SCN5A; FIG. 11), with the sodium channel pore and selectivity filter inducing a greater disease burden as previously observed. FIG. 12 examines in greater detail a small region within domain III (D-III), showing the 95% credible interval of BrS1 penetrance both before (prior) and after (posterior) adding heterozygote counts listed on the left. The selectivity filter has the highest average BrS1 prior and posterior, also true for domains I, II, and IV (FIG. 11). Towards the intracellular side of the D-III S6 helix, there are fewer variants with high BrS1 penetrance. This trend can also be seen in FIG. 13 which shows an increase in variants associated with BrS1 that depends on membrane depth of the variant. These results support our assertion that variant-specific predictive features of variant-induced functional perturbation and structural context contain information equivalent to clinically phenotyping individuals heterozygous for these variants.

A modified Bayesian approach to estimate BrS penetrance. An Empirical Bayes approach combines information across all variants to estimate a single prior distribution and estimate a variant-specific posterior penetrance from that prior. These estimates assume all variant effects have the same prior and therefore shrink towards a global mean across all variants. Here we put forward a method to model the penetrance for each variant using variant-specific predictive features. The resulting penetrance and uncertainty estimates yield a posterior that can be re-used as variant-specific prior (interpretable as equivalent to hypothetical observations of affected and unaffected heterozygotes) in a Bayesian updating scheme. This information is accessible before clinically phenotyping a single heterozygote; example estimates of high BrS1 penetrance [c.4213G>C (p.Val1405Leu), c.4259G>T (p.Gly1420Val), and c.4258G>C (p.Gly1420Arg)] and low BrS1 penetrance [c.4418T>G (p.Phe1473Cys), c.4459A>C (p.Met1487Leu), and c.4467G>T (p.Glu1489Asp)] are seen in FIG. 12.

Comparison between penetrance prediction and ACMG variant classification. We put forward a method to estimate the probability that a SCN5A variant will manifest in BrS1 for a given patient (our ‘risk score’), and uncertainty for that score, conditioned on variant attributes. We are not assessing the causality of the variant and its attributes on the manifestation of disease, but rather their association. Hence, our framework diverges from that of the ACMG. For example, in our formulation, a VUS with many affected heterozygotes would have the same probability distribution as a pathogenic variant with many affected heterozygotes [provided the number of observations of cases and controls is the same and the other predictive covariates (variant attributes) are the same]. If there are comparatively few heterozygotes of the VUS, given the same predictive covariates, greater uncertainty would be reflected by a wider distribution of penetrance probability (FIG. 8). In addition, our calculation is agnostic to origin, de novo or inherited, and therefore does not consider this evidence (though this information may additionally inform an estimate of penetrance and therefore warrants further investigation). We also do not treat null variants here. For our purposes of building variant-specific, data-driven penetrance priors, null variants have relatively little variance in the predictive covariates and therefore contribute less to our analysis. In future work we will additionally attempt to include these features.

Prospects for applications of this method. Our approach provides a risk score for disease, in this case, for BrS1. However, Brugada syndrome has degrees of electrophysiologic phenotypes and symptoms. We envision being able to predict these degrees of clinical phenotype from variant-specific properties in the future by integrating electronic health records with linked genetic data. However, at present, these granular electrophysiologic and symptom data are not available for a number of unique heterozygotes and unique variants sufficient for statistical analysis. Beyond SCN5A and BrS1, a reasonable next step would involve the 59 genes for which the ACMG recommends clinical diagnostic laboratories report secondary variant discovery. Of these, 36 have greater than or equal to 20 missense “pathogenic”/“likely pathogenic” variants in ClinVar, suggesting that many variants are described in the literature and can be curated in a similar manner to SCN5A. It is also important to note that the penetrance estimates derived in our approach are not static and will continue to be refined as additional data become available, i.e. phenotype data from case reports and large biobank projects, additional in vitro functional studies, and improved computational and structural predictors.

Our approach provides a risk score for disease, in this case BrS1, analogous to a diagnostic test (might patient X develop BrS1 given they have variant Y). If we know patient X already has BrS1, we can use their data to inform other individuals' risk scores, but we cannot use our approach to absolutely determine the role of variant Y manifesting disease. One application of our approach is that we can examine the ratio P(BrS1|Variant X)/P(BrS1|wild-type) to see if the data better support that variant X is on the causal pathway to disease. But we caution that this approach is imperfect; it does not allow for variants to interact, for example. Additionally, while clinical evidence affirms a strong relationship between SCN5A variants and BrS1, many genetic and environmental factors influence the ultimate presentation of BrS1 in an individual. Not accounting for additional demographic, genetic, or environmental factors certainly increased the noise in our analysis. To counter this as best as possible, we included the maximum number of carriers for the maximum number of unique variants. Finally, we recognize the likely bias intrinsic to compiling a list of affected and unaffected heterozygotes in the manner outlined in the methods section above; however, the most probable manifestation of these biases would be the loss of an observable relationship between the predictive features and penetrance, not the creation of a spurious relationship.

We advance a method to estimate a degree of clinical heterogeneity in variant impact, incomplete penetrance. Here we have demonstrated how BrS1 penetrance can be estimated with high accuracy and precision. Using a Bayesian framework to estimate penetrance allows us to quantitatively integrate clinical phenotypic data with variant-specific functional measurements, variant classifiers, and sequence- and structure-based features to accurately estimate penetrance. This method can be extended to other genes and disorders in order to enable quantitative interpretation of variants probabilistically and quantitatively.

Applying the method of FIG. 1 specifically to the SCN5A gene, where individual variants are known to influence clinical presentation of the autosomal dominant arrhythmia Brugada Syndrome (BrS1), we define cases as individuals with either a spontaneous or drug-induced ECG BrS1 patterns, ST-segment abnormalities. We apply equation (1) where a is the number of variant heterozygotes diagnosed with BrS1 (or BrS1 cases) and β is the number of unaffected heterozygotes of the same variant (or controls). As the total number of observed heterozygotes increases, the estimated penetrance converges to the traditional definition. The mean posterior penetrance can be thought of as a shrunken estimate of the observed penetrance, especially for variants with small numbers of known heterozygotes.

To generate priors from our available data, we use the expectation maximization (EM) algorithm described above in reference to FIG. 1. As discussed above, the EM algorithm is an iterative technique composed of three steps: 1) calculate the expected penetrance from an empirical Bayes penetrance model, 2) fit a regression model of our estimated penetrance on variant-specific characteristics by maximum likelihood (Equation (2)) and 3) revise our estimate of the BrS1 penetrance prior using fit from step 109 then iterate steps 109 through 113 until convergence criteria are satisfied (e.g., step 115).

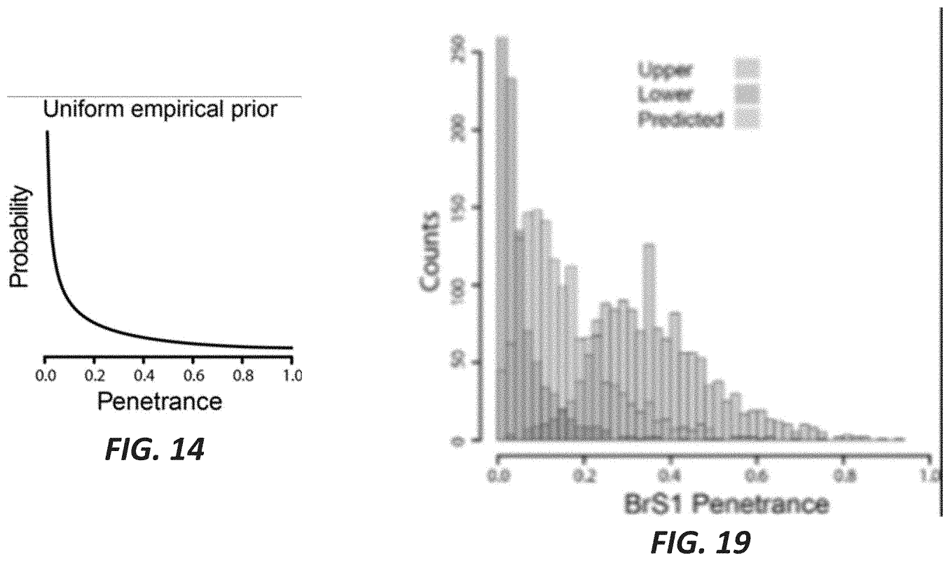

In this example using equation (2) specifically for BrS1 penetrance, equation (2) peak current is an in vitro measurement of the maximum current through a channel (normalized to wild type), penetrance density is a structure-based metric (as described in further detail below), and in silico variant-classifiers is a vector populated with commonly used variant classification servers such as PROVEAN and PolyPhen (see below); all predictors used are continuous, not categorical or binary (in the table of FIG. 7). The fitted model is then used to generate an updated prior distribution and, by addition of observed cases and controls for each variant, a subsequent posterior expected penetrance. The updated posterior penetrance is then used to build a new fitted model and further refine the posterior expected penetrance. This procedure is iterated until it converges to the maximum likelihood solution (using the method of FIG. 1). Using a beta-binomial model to estimate penetrance, the prior parameters (α_{prior, EM} and β_{prior, EM}, both functions of the features listed in the table of FIG. 7) are identifiable from a predicted penetrance and its associated variance. For comparison, we generated predicted penetrance values using a standard empirical Bayes method which generated a single empirical prior for all variants, α_{prior, empirical} and β_{prior, empirical} equal to 0.45 and 2.73, respectively (called empirical prior throughout the text, see, e.g. FIG. 14). To test our predictions, we compare our EM penetrance priors,

$$\frac{{α}_{prior , EM}}{{α}_{prior , EM} + {β}_{prior , EM}} ,$$

to the posterior mean penetrance derived by adding BrS1 cases and controls for each variant to the empirical prior,

$$\frac{BrS 1 cases + {α}_{prior , empirical}}{Total heterozygotes + {α}_{prior , empirical} + {β}_{prior , empirical}} ,$$

or the EM prior,

$$\frac{BrS 1 cases + {α}_{prior , EM}}{Total heterozygotes + {α}_{prior , EM} + {β}_{prior , EM}} .$$

Collection of the SCN5A variant dataset. The dataset was curated from 711 papers in a previous publication, to which we added an additional 45 papers on SCN5A that had been published since the previous dataset was constructed. Briefly, we searched publications for the number of heterozygotes of each variant mentioned, the number of unaffected and affected individuals with diagnosed BrS1, and variant-induced changes in channel function, if reported; all recorded values were normalized to wild-type values reported in the same publications. We supplemented this dataset with all SCN5A variants in the gnomAD database of population variation (http://gnomad.broadinstitute.org/; release 2.0). Due to the rarity of BrS1 (-1 in 10,000), all heterozygotes found in gnomAD were counted as unaffected. An interactive version of the dataset, the SCN5A Variant Browser, is available at http://oates.mc.vanderbilt.edu/vancart/SCN5A/. We further collected in silico pathogenicity predictions from three commonly used servers: SIFT, Polyphen-2, and PROVEAN. We also include basic local alignment search tool position-specific scoring matrix (BLAST-PSSM) for SCN5A and the per residue evolutionary rate, previously shown to have predictive value for predicting functional perturbation for the cardiac potassium channel gene KCNQ1, and point accepted mutation score (PAM). Additionally, we leveraged structures of the SCN5A protein product and derived a penetrance density as previously described (as described further below). In-frame indels are treated as missense variants. We include these variants as variations at a residue where the indel starts, and only note whether they are an insertion or deletion. Some of these variants have functional data available and their penetrance densities are calculated from the residue starting the indel. These are simplifications to enable an analysis of as many variants and heterozygote individuals as possible. For these variants, we did not include in silico pathogenicity predictions. We included compound heterozygotes (individuals with more than one SCN5A variant) as separate records when these data are available, though these were very rare. Additionally, our inclusion criteria are not modified by relatedness. We did not include intronic variants in our analysis.

Initial Empirical Bayes beta-binomial prior penetrance calculation. Using the data from the aforementioned literature curation, we estimated the penetrance for each observed variant using a beta-binomial empirical Bayes model. To calculate the empirical BrS1 penetrance prior, we calculated α_{prior, empirical} and β_{prior, empirical} by finding the weighted mean penetrance over all variants in the dataset and estimating the variance. Weighting was done using the following equation:

$$w = 1 - \frac{1}{0.01 + number of heterozygotes} \qquad ( 3 )$$

Equation 3 ensures variants with a greater number of total heterozygotes (and therefore higher confidence in penetrance estimate) had a greater weight in the preliminary analysis. We then estimated the variance in penetrance as the mean squared error (MSE) between the estimated penetrance mean and the observed penetrance from Equation 1 with αₚᵣᵢₒᵣ and βₚᵣᵢₒᵣ equal to zero. With these estimated mean and MSE-derived variance, the empirical prior penetrance was calculated to be an αₚᵣᵢₒᵣ and βₚᵣᵢₒᵣ equal to 0.45 and 2.73, respectively. The variant-specific empirical posterior for each variant was then calculated by adding observed heterozygote counts of affected (BrS1 cases) and unaffected to α_{prior, empirical} and β_{prior, empirical} respectively, and the resulting posterior mean penetrance was used as the dependent variable of the subsequent regression model (Equation 2). The inverse variance of the estimated posterior beta distributions capped at the ninth decile determined in this step were used to weight subsequent regression models and Pearson R² calculations.

Expectation maximization Bayesian beta-binomial penetrance predictions. To deal with missing data in a prediction model, we followed an approach which avoids multiple imputation but guarantees maximum predictive accuracy across missing data patterns. In short, for every missing data pattern, we estimate a separate prediction model. For example, His558Arg, where penetrance density, in silico predictors, and functional data are all available, the estimate of penetrance is regressed on all other variants where all of these covariates are available (n=238). For Tyr1449Cys, however, only penetrance density and in silico predictors are available, so only those covariates are used in the regression (n=1,382; much higher since functional data have been collected for relatively few variants). The models were built with a linear regression pattern-mixture algorithm, updating posterior mean penetrances iteratively until the resulting estimated mean penetrance,

$$μ = \frac{{α}_{p r i o r , E M}}{{α}_{p r i o r , E M} + {β}_{p r i o r , E M}} ,$$

changed by <0.01% from the previous iteration. This process typically converged within eight iterations. For variant, i, the variance was estimated from this converged EM mean penetrance according to (Equation 4):

$${σ}_{i} = \frac{{μ}_{i} ( 1 - {μ}_{i} )}{1 + v} \qquad ( 4 )$$

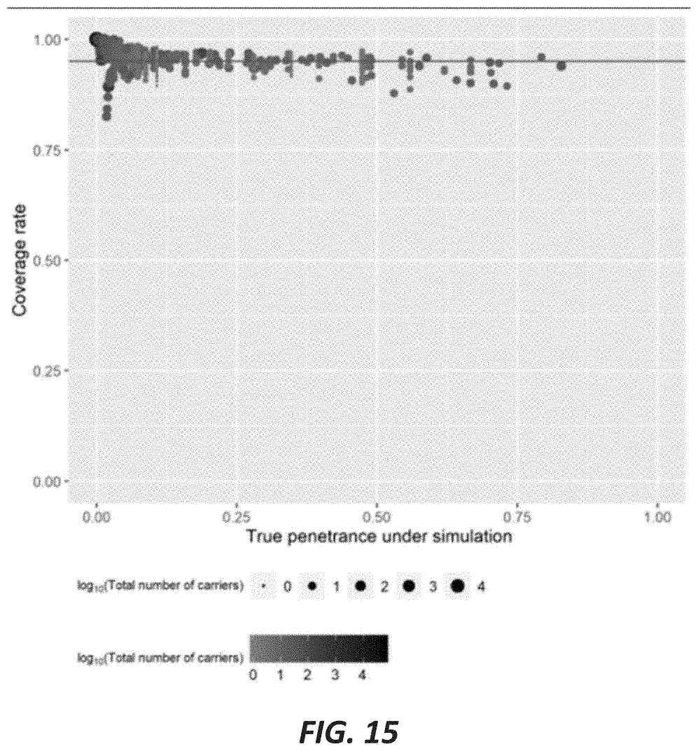

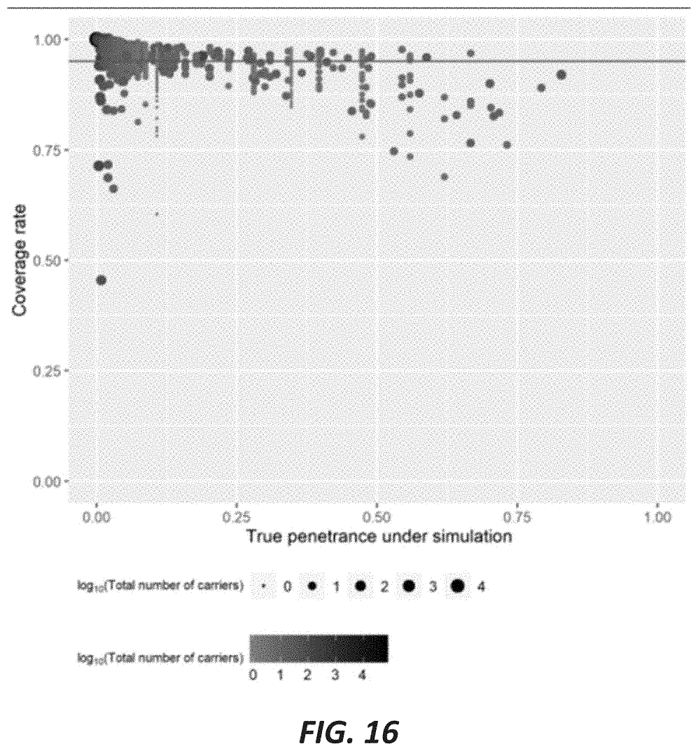

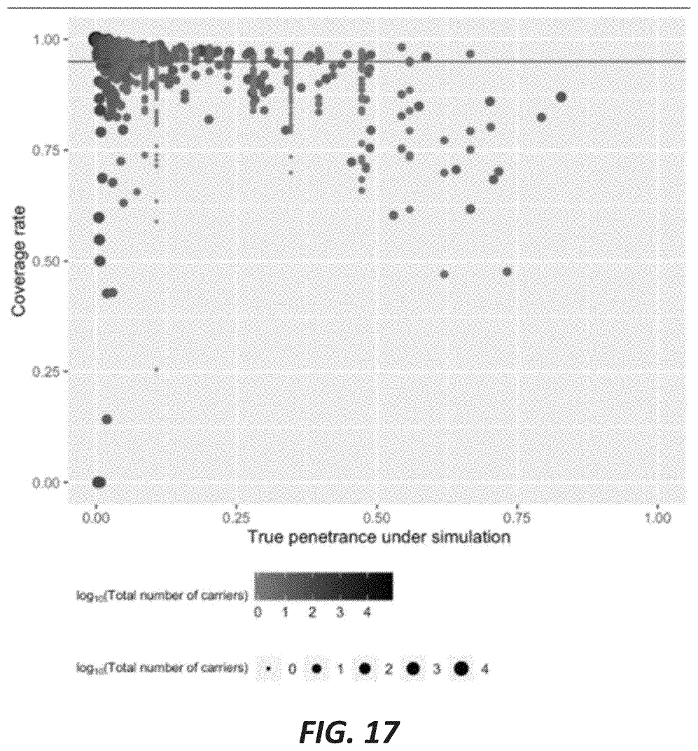

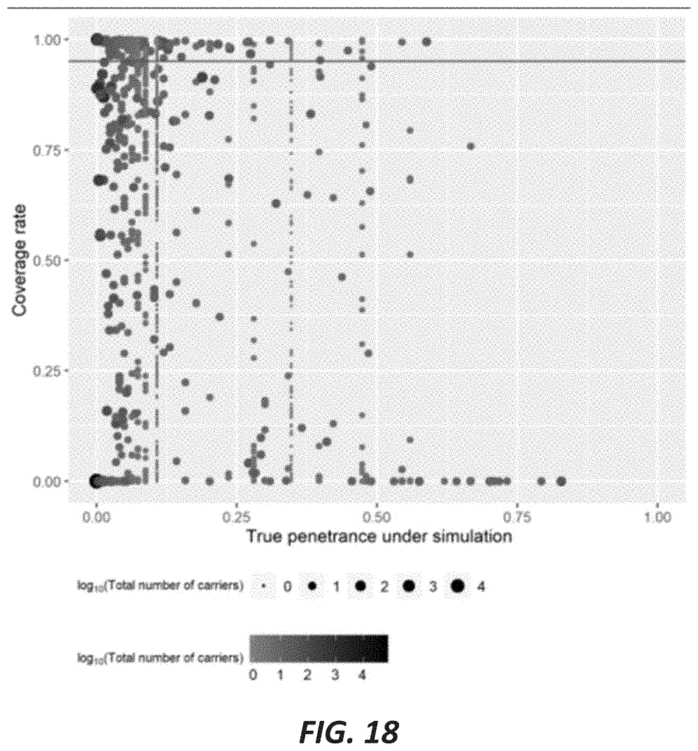

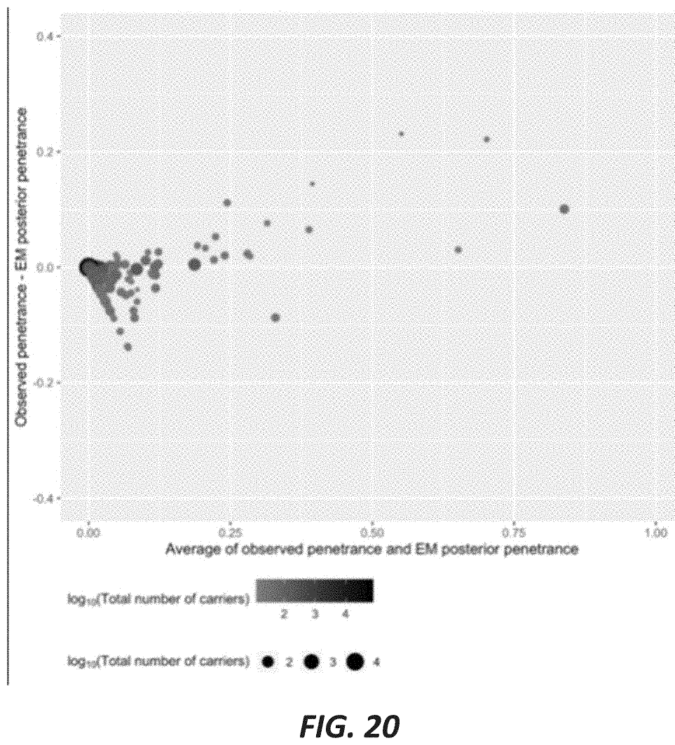

We then adjusted v, equivalent to the number hypothetical observations of clinically phenotyped heterozygotes, to balance overcoverage of variants with low to moderate BrS1 penetrance and poorer coverage of variants with high estimated mean penetrance, resulting in a range of v, from approximately 15 to 20 (see FIGS. 15-18).

As discussed above, one variant-specific feature that may be used in some implementations to estimate the disease penetrance for the specific variant is “penetrance density.” In some implementations, “penetrance densities” are calculated (for BrS1 or for other genes/variants) based on an approach akin to k-nearest neighbors. We average empirical BrS1 mean posterior penetrance

$$( \frac{BrS 1 cases + {α}_{prior , empirical}}{Total carriers + {α}_{prior , empirical} + {β}_{prior , empirical}} )$$

of variants weighted by the inverse distance in space from the variant of interest. This calculated feature therefore depends on how many variants are near the variant of interest, with regions in three-dimensional space dense with high penetrance variants—“hotspots”—yielding a higher penetrance prediction. Penetrance density is calculated as follows:

$${ρ}_{j} = \frac{{\sum }_{i = 0}^{n} BrS 1 Posterior {Penetrance}_{i , mutation ( i \neq j )} \cdot \frac{1}{1 + {e}^{( \frac{{d}_{i , j}}{2} )}}}{{\sum }_{i = 0}^{n} \frac{1}{1 + {e}^{( \frac{{d}_{i , j}}{2} )}}} \qquad ( 5 )$$

where ρⱼ is BrS1 penetrance density of the j^{th} variant, BrS1 Posterior Penetrance_{x,i,mutation(i≠j)} is the empirical BrS1 mean posterior penetrance for the i^{th} variant, and d_{i,j} is the distance between the center of mass of residues i and j. i does include residue j, but only if the identity of the amino-acid mutation is changed, i.e. mutation(i)≠mutation(j). For residues in the flexible linkers where structural data are not available, we assumed a flexible amino-acid polymer and calculated the distance between residues as d_{i,j}=3.8*\sqrt{R_{i,j}}, where R_{i,j} is the number of residues between residue i and residue j.

Also, as discussed above in reference to equation (4), we scaled the variance from the convergent EM result by a factor of “v.” At each level of ‘v’ and for each variant, we sampled from binomial distribution with n of 100, and probability of

$$\frac{BrS 1 cases + {α}_{prior , empirical}}{total carriers + {α}_{prior , empirical} + {β}_{prior , empirical}}$$

We calculated the resulting 95% posterior credible interval from the Beta distribution with shape parameters 1) BrS1 sampled+α_{prior,EM} and 2) 100−BrS1 sampled+β_{prior,EM}. We repeated this process 1000 times, and calculated the rate of the posterior credible interval covering the probability

$$\frac{BrS 1 cases + {α}_{prior , empirical}}{total carriers + {α}_{prior , empirical} + {β}_{prior , empirical}} .$$

We selected the best ‘v’ from the coverage plots which balances the tradeoff of over-coverage in variants with medium low BrS1 penetrance and under-coverage of variants with high BrS1 penetrance. From this procedure we chose a tuning parameter of ‘v’=19 (see, e.g., FIGS. 13, 14, and 19).

Accordingly, the systems and methods described herein provide mechanisms for estimating variant-specific disease penetrance and then assigning a classification of disease risk for patients exhibiting the variant based on the variant-specific disease penetrance. In some implementations, the systems and methods described herein provide automated mechanisms for detecting occurrences of a specific variant in electronic medical records and for then taking mitigative action (e.g., notifying a medical professional and/or initiating medical treatment) based on the classification of disease risk assigned to the variant based on the variant-specific disease penetrance. Further features and advantages are set forth in the accompanying claims.

What is claimed is:

1. A method of assessing a probability of a disease occurring in a patient, the method comprising:
selecting a set of variants including a specific variant of interest;
accessing, by a computer-based system, a database including genetic and disease data for each of a plurality of individuals;
calculating an empirical prior estimate of disease penetrance based on
a number of individuals from the plurality of individuals that have both the disease and at least one variant of the set of variants, and
a number of individuals from the plurality of individuals that have at least one variant of the set of variants;
calculating a posterior penetrance estimate for the specific variant of interest based on the empirical prior estimate, a number of individuals from the plurality of individuals that have both the disease and the specific variant of interest, and a number of individuals from the plurality of individuals that have the specific variant of interest;
applying, by the computer-based system, a recursive regression modeling to the posterior penetrance estimate, wherein the recursive regression modeling includes
fitting an estimated penetrance for the specific variant of interest to the posterior penetrance estimate,
defining a set of revised variant-specific priors based on the fitting,
recalculating the posterior penetrance estimate based on the set of revised variant-specific priors, and
terminating the recursive regression modeling in response to determining that a most recent iteration of the recursive regression modeling satisfies one or more defined exit criteria; and
determining the probability of the disease occurring in a patient that has the specific variant of interest based on the posterior penetrance estimate as determined by the recursive regression modeling.

2. The method of claim 1, wherein calculating the posterior penetrance estimate includes calculating the posterior penetrance estimate as
$$posterior penetrance {estimate}_{i} = \frac{{α}_{i} + {α}_{prior}}{{α}_{i} + {α}_{prior} + {β}_{i} + {β}_{prior}}$$
wherein αᵢ is the number of individuals from the plurality of individuals that have both the disease and the specific variant of interest, βᵢ is the number of individuals from the plurality of individuals that have the specific variant of interest and not the disease,
$$\frac{{α}_{prior}}{{α}_{prior} + {β}_{prior}}$$
is a mean disease penetrance observed across all variants of the set of variants, αₚᵣᵢₒᵣ is a Bayesian prior that biases the posterior penetrance estimate towards the mean disease penetrance observed across all variants of the set of variants, and βₚᵣᵢₒᵣ is another Bayesian prior that biases the posterior penetrance estimate towards the mean disease penetrance observed across all variants of the set of variants.

3. The method of claim 2, wherein recalculating the posterior penetrance estimate based on the set of revised variant-specific priors includes recalculating the posterior penetrance estimate as
$$posterior penetrance {estimate}_{i} = \frac{{α}_{i} + {α}_{i , prior}}{{α}_{i} + {α}_{i , prior} + {β}_{i} + {β}_{i , prior}}$$
wherein α_{i,prior} is an updated estimate of individuals having both the specific variant in questions and the disease based on the iteration of the recursive regression modeling, and β_{i,prior} is an updated estimate of individuals having the specific variant in question and not the disease based on the iteration of the recursive regression modeling.

4. The method of claim 1, wherein determining that the most recent iteration of the recursive regression modeling satisfies the one or more defined exist criteria includes determining that a mean penetrance calculated by the posterior penetrance estimate has changed by less than 10% as a result of the most recent iteration.

5. The method of claim 1, wherein applying, by the computer-based system, the recursive regression modeling to the posterior penetrance estimate includes applying a linear regression modeling with an expectation maximization.

6. The method of claim 1, wherein determining the probability of the disease occurring in the patient that has the specific variant of interest includes
assigning to the specific variant of interest a relative classification of disease probability based on the posterior penetrance estimate, and
assigning to a patient having the specific variant of interest a probability of the disease occurring based on the relative classification of disease probability assigned to the specific variant of interest.

7. The method of claim 6, wherein assigning to the specific variant of interest the relative classification of disease probability includes assigning a relative classification selected from a group consisting of benign, mild risk, and pathogenic.

8. The method of claim 1, wherein determining the probability of the disease occurring in the patient includes:
automatically searching, by the computer-based system, a set of electronic health records for occurrences of the specific variant of interest, and
notifying a health care provider for each patient with a detected occurrence of the specific variant of interest of the probability of disease occurring for each detected occurrence of the specific variant of interest by at least one selected from a group consisting of updating an electronic health record to include an indication of the determined probability of the disease associated with the specific variant of interest and transmitting a notification to the health care provider including the indication of the determined probability of the disease associated with the specific variant of interest.

9. A system for assessing a probability of a disease occurring in a patient, the system comprising an electronic controller configured to:
select a set of variants including a specific variant of interest;
access a database including genetic and disease data for each of a plurality of individuals;
calculate an empirical prior estimate of disease penetrance based on
a number of individuals from the plurality of individuals that have both the disease and at least one variant of the set of variants, and
a number of individuals from the plurality of individuals that have at least one variant of the set of variants;
calculate a posterior penetrance estimate for the specific variant of interest based on the empirical prior estimate, a number of individuals from the plurality of individuals that have both the disease and the specific variant of interest, and a number of individuals from the plurality of individuals that have the specific variant of interest;
apply a recursive regression modeling to the posterior penetrance estimate, wherein the recursive regression modeling includes
fitting an estimated penetrance for the specific variant of interest to the posterior penetrance estimate,
defining a set of revised variant-specific priors based on the fitting,
recalculating the posterior penetrance estimate based on the set of revised variant-specific priors, and
terminating the recursive regression modeling in response to determining that a most recent iteration of the recursive regression modeling satisfies one or more defined exit criteria; and
determine the probability of the disease occurring in a patient that has the specific variant of interest based on the posterior penetrance estimate as determined by the recursive regression modeling.

10. The system of claim 9, wherein the electronic controller is configured to calculate the posterior penetrance estimate by calculating the posterior penetrance estimate as
$$posterior penetrance {estimate}_{i} = \frac{{α}_{i} + {α}_{prior}}{{α}_{i} + {α}_{prior} + {β}_{i} + {β}_{prior}}$$
wherein αᵢ is the number of individuals from the plurality of individuals that have both the disease and the specific variant of interest, βᵢ is the number of individuals from the plurality of individuals that have the specific variant of interest and not the disease,
$$\frac{{α}_{prior}}{{α}_{prior} + {β}_{prior}}$$
is a mean disease penetrance observed across all variants of the set of variants, αₚᵣᵢₒᵣ is a Bayesian prior that biases the posterior penetrance estimate towards the mean disease penetrance observed across all variants of the set of variants, and βₚᵣᵢₒᵣ is another Bayesian prior that biases the posterior penetrance estimate towards the mean disease penetrance observed across all variants of the set of variants.

11. The system of claim 10, wherein the electronic controller is configured to recalculate the posterior penetrance estimate based on the set of revised variant-specific priors by recalculating the posterior penetrance estimate as
$$posterior penetrance {estimate}_{i} = \frac{{α}_{i} + {α}_{i , prior}}{{α}_{i} + {α}_{i , prior} + {β}_{i} + {β}_{i , prior}}$$
wherein α_{i,prior} is an updated estimate of individuals having both the specific variant in questions and the disease based on the iteration of the recursive regression modeling, and β_{i,prior} is an updated estimate of individuals having the specific variant in question and not the disease based on the iteration of the recursive regression modeling.

12. The system of claim 9, wherein the electronic controller is configured to determine that the most recent iteration of the recursive regression modeling satisfies the one or more defined exist criteria by determining that a mean penetrance calculated by the posterior penetrance estimate has changed by less than 10% as a result of the most recent iteration.

13. The system of claim 9, wherein the electronic controller is configured to apply the recursive regression modeling to the posterior penetrance estimate by applying a linear regression modeling with an expectation maximization.

14. The system of claim 9, wherein the electronic controller is configured to determine the probability of the disease occurring in the patient that has the specific variant of interest by
assigning to the specific variant of interest a relative classification of disease probability based on the posterior penetrance estimate, and
assigning to a patient having the specific variant of interest a probability of the disease occurring based on the relative classification of disease probability assigned to the specific variant of interest.

15. The system of claim 14, wherein the electronic controller is configured to assign to the specific variant of interest the relative classification of disease probability by assigning a relative classification selected from a group consisting of benign, mild risk, and pathogenic.

16. The system of claim 9, wherein the electronic controller is configured to determine the probability of the disease occurring in the patient by:
automatically searching a set of electronic health records for occurrences of the specific variant of interest, and
notifying a health care provider for each patient with a detected occurrence of the specific variant of interest of the probability of disease occurring for each detected occurrence of the specific variant of interest by at least one selected from a group consisting of updating an electronic health record to include an indication of the determined probability of the disease associated with the specific variant of interest and transmitting a notification to the health care provider including the indication of the determined probability of the disease associated with the specific variant of interest.
